(19) United States
(12) Patent Application Publication
(10) Pub. No.: US 2012/0321724 A1
(43) Pub. Date: Dec. 20, 2012

(54) Method of Measuring and Monitoring In Vivo Nitrite Levels

(76) Inventor: Nathan S. Bryan, Houston, TX (US)

(21) Appl. No.: 13/584,306

(22) Filed: Aug. 13, 2012

Related U.S. Application Data
(63) Continuation-in-part of application No. 12/484,364, filed on Jun. 15, 2009, now Pat. No. 8,298,589.
(60) Provisional application No. 61/522,498, filed on Aug. 11, 2011.

(51) Int. Cl. A61K 33/00; G01N 33/50; A61P 9/10; A61P 3/10; A61P 3/06; A61P 9/12; G01N 33/52; C12Q 1/12

(52) U.S. Cl. 424/718; 436/110; 435/37

(57) ABSTRACT

A method of measuring in vivo nitric oxide and nitrite levels in individuals by providing a salivary nitrite test substrate, testing salivary nitrite levels with the test substrate, measuring nitrite levels detected in the testing; and correlating the measured nitrite levels with in vivo nitric oxide bio-availability.

This application claims priority to U.S. Provisional Patent Application Ser. No. 61/522,498, filed Aug. 11, 2011, and is a continuation in part of U.S. patent application Ser. No. 12/484,364, filed Jun. 15, 2009, both of which are incorporated by reference herein in their entirety.

### TECHNICAL FIELD

The present invention generally concerns at least the fields of biology, cell biology, molecular biology, medical analytics, and medicine.

### BACKGROUND OF THE INVENTION

The production of nitric oxide (NO) is one of the most important biological processes in our body. Endothelial dysfunction is a physiological dysfunction of normal biochemical processes carried out by the endothelium, the cells that line the inner surface of all blood vessels including arteries and veins (as well as the innermost lining of the heart and lymphatics). Loss of endothelial NO function is associated with several cardiovascular disorders, including atherosclerosis, which is due either to decreased production or to increased degradation of NO. Experimental and clinical studies provide evidence that defects of endothelial NO function, referred to as endothelial dysfunction, is not only associated with all major cardiovascular risk factors, such as hyperlipidemia, diabetes, hypertension, smoking and severity of atherosclerosis, but also has a profound predictive value for the future atherosclerotic disease progression. The dysfunctional eNOS/NO pathway is considered as an early marker or a common mechanism for various cardiovascular disorders. Over the last two decades, it has become evident that decreased bioavailability of endothelial NO, produced from endothelial NO synthase (eNOS), plays a crucial role in the development and progression of a number of human diseases. As we age, we lose our ability to generate NO and as a result our risk of developing heart or vascular disease increases. Most people are unaware of their nitric oxide status. This is primarily because there currently does not exist an easy to use, non-invasive standard diagnostic for NO availability in humans.

The only true measure of endothelial function and NO production is through flow-mediated dilatation studies that can only be performed in a clinical setting. Therefore developing an easy to use, non-invasive test, such as a colorimetric test, for assessing NO status will help people realize their deficiency and undertake strategies or lifestyle modifications to restore NO homeostasis that may help prevent the onset and development of cardiovascular disease.

For the past 20 years, scientists have used plasma nitrite as a marker of acute nitric oxide production. Although detection methods exist for quantifying nitrite in biological samples, this still is not part of the laboratory analysis of diagnostic labs that perform the majority of the blood labs in the U.S.

In addition to the endogenous oxidation of NO, nitrite is also derived from reduction of salivary nitrate by commensal bacteria in the mouth and gastrointestinal tract as well as from dietary sources such as meat, vegetables and drinking water.

The bioactivation of nitrate from dietary or endogenous sources requires its initial reduction to nitrite, and because mammals lack specific and effective nitrate reductase enzymes, this conversion is mainly carried out by commensal bacteria in the mouth and gastrointestinal tract and on body surfaces. Nitrate from the diet is rapidly absorbed in the upper gastrointestinal tract. In the blood, it mixes with the nitrate formed from the oxidation of endogenous NO produced from the NOS enzymes. Through early cancer research, it was known that up to 25% of circulating nitrate is actively taken up by the salivary glands and concentrated 10- to 20-fold in saliva, but the reason and mechanism for this were unknown, other than its proposed pathologic role in formation of carcinogenic nitrosamines.

After ingestion of nitrate and effective absorption in the upper gastrointestinal tract, salivary concentrations of nitrate become very high (millimolar). In the oral cavity, commensal facultative anaerobic bacteria, located in the deep crypts of the posterior part of the tongue, reduce nitrate to nitrite by action of nitrate reductase enzymes. These bacteria use nitrate as an alternative terminal electron acceptor during respiration to gain adenosine-5_-triphosphate (ATP) in the absence of oxygen. When swallowed saliva meets the acidic gastric milieu, part of the nitrite is immediately protonated to form nitrous acid (HNO₂), which then decomposes to nitric oxide and other nitrogen oxides. Concentrations of nitric oxide gas in the stomach can be substantial (more than 100 ppm) and sometimes beyond what is considered safe as a working environment by the authorities. Most of the salivary nitrite escapes the gastric conversion to nitric oxide and enters the systemic circulation.

Human nitrate reduction is highly dependent on the oral commensal bacteria, because our cells do not convert nitrate to nitrite to a high degree. This is evident by studies where the biologic effects of ingested nitrate, as well as the concomitant increase in plasma nitrite, are abolished after avoiding swallowing of saliva or by the use of an antibacterial mouthwash. Moreover, germ-free mice have virtually no gastric nitric oxide, even after a nitrate load.

After a meal rich in nitrate, the levels in plasma increase greatly and remain high for a prolonged period of time (plasma half-life of nitrate is 5-6 hours). The nitrite levels in plasma also increase after nitrate ingestion. Human nitrate reduction requires the presence of these bacteria—suggesting a functional symbiosis relationship—as mammalian cells cannot effectively metabolize this anion.

The salivary nitrate levels can approach 10 mM and nitrite levels 1-2 mM after a dietary nitrate load. When saliva enters the acidic stomach (1-1.5 L per day), much of the nitrite is rapidly protonated to form nitrous acid (HNO₂; pKa 3.3), which decomposes further to form NO and other nitrogen oxides. Once nitrite is absorbed and circulated, it is taken up by peripheral tissues and can be stored in cells. The one-electron nitrite reduction to NO can occur in a much simpler mechanism than the two-electron reduction of nitrate by bacteria.

The 1-electron reduction of nitrite can occur by ferrous heme proteins (or any redox active metal) through the following reaction:

NO2-+Fe(II)+H+∇NO+Fe(III)+OH-

This is the same biologically active NO as that produced by NOS, with nitrite rather than L-arginine as the precursor and is a relatively inefficient process. Much of the recent focus on nitrite physiology is due to its ability to be reduced to NO during ischemic or hypoxic events. Nitrite reductase activity in mammalian tissues has been linked to the mitochondrial electron transport system, protonation, deoxyhemoglobin, and xanthine oxidase. Nitrite can also transiently form nitrosothiols (RSNOs) under both normoxic and hypoxic conditions and a recent studies demonstrate that steady state concentrations of tissue nitrite and nitroso are affected by changes in dietary NOx (nitrite and nitrate) intake. Furthermore enriching dietary intake of nitrite and nitrate translates into significantly less injury from heart attack.

Previous studies also demonstrated that nitrite therapy given intravenously prior to reperfusion protects against hepatic and myocardial I/R injury. Additionally, experiments in primates revealed a beneficial effect of long-term application of nitrite on cerebral vasospasm. Moreover, inhalation of nitrite selectively dilates the pulmonary circulation under hypoxic conditions in vivo in sheep. Topical application of nitrite improves skin infections and ulcerations. Furthermore, in the stomach, nitrite-derived NO seems to play an important role in host defense and in regulation of gastric mucosal integrity. All of these studies together along with the observation that nitrite can act as a marker of NOS activity opened a new avenue for the diagnostic and therapeutic application of nitrite, especially in cardiovascular diseases, using nitrite as marker as well as an active agent. Oral nitrite has also been shown to reverse L-NAME induced hypertension and serve as an alternate source of NO in vivo. Studies demonstrate that plasma nitrite levels progressively decrease with increasing cardiovascular risk. Since a substantial portion of steady state nitrite concentrations in blood and tissue are derived from dietary sources, modulation of nitrite and/or nitrate intake may provide a first line of defense for conditions associated with NO insufficiency. In fact it has been reported that dietary nitrate reduces blood pressure in healthy volunteers.

### BRIEF SUMMARY OF THE INVENTION

The present invention concerns methods and compositions for measuring in vivo nitric oxide (NO) and nitrite levels in a mammal. Such methods and compositions utilize a test substrate that when exposed to oral samples, such as exposed to saliva and/or breath, provides determination of a level of NO and/or nitrite in the body. In particular cases the oral amount of NO and/or nitrite that is detected is representative of the total amount of same in the body.

In some embodiments of the invention, there is a method of measuring in vivo nitric oxide and nitrite levels in humans comprising providing a salivary nitrite test strip; testing salivary nitrite levels with said test strip; measuring nitrite levels detected in said testing; correlating said measured nitrite levels with in vivo nitrite levels; and determining specific activity of nitrate reducing bacteria in the mouth. In specific aspects, a baseline salivary nitrite level is determined by an initial measurement after a 3-5 hour fast. In some embodiments, the measuring is by colormetric indicia.

In certain embodiments of the invention, there is a method of monitoring in vivo nitric oxide or nitrite levels in humans comprising a method of the invention; and repeating the method at regular intervals, such as twice daily measurements. In some embodiments, the regular intervals include a test 1.5-3 hours after a nitrate rich meal. In specific cases, the method comprises testing 3-4 hours after exercise. In specific embodiments, the method further comprises testing 3-4 hours after ingesting nitrate rich foods.

In some embodiments of the invention, there is a method of monitoring and adjusting in vivo nitric oxide and nitrite levels comprising performing a method of the invention; recording test results from multiple days; and adjusting intake of nitrate, nitrite, or related compounds based on the test results.

In some embodiments of the invention, there is a method of monitoring and determining nitrate-reducing bacteria in the oral cavity of an individual. In specific cases, the method can measure nitrite formation after giving a nitrate load and/or supplement in the oral cavity (for example) and determine the presence and/or activity of nitrate-reducing bacteria in the oral cavity. The presence or absence of nitrate-reducing bacteria can be an additional risk factor for cardiovascular disease because of the formation of nitrite and nitric oxide, in at least certain aspects.

In some embodiments of the invention, there is a method of determining the level of orally-produced nitric oxide (NO) or nitrite in an individual comprising the steps of providing an oral nitrite test substrate; and exposing said test substrate to an oral sample, such as saliva or breath, to produce a measured test level of NO or nitrite. The method may further comprise the step of correlating the measured test level with in vivo NO or nitrite level in the individual. In specific embodiments, the method is performed after fasting for at least three hours. In at least certain cases, the measured test level is generated with colorimetric indicia. In some embodiments, the method is performed for the individual more than once and may be performed for the individual at regular intervals, such as once or twice daily measurements, for example. The method may be performed following subjecting the individual to conditions that increase the in vivo level of NO or nitrite. In some embodiments, the condition comprises consumption of a nitrate-rich food and/or beverage, and in certain cases the method is performed at least 1.5 hours after consumption of the food and/or beverage. In specific embodiments, the condition comprises exercise, and in at least specific cases the method is performed at least three hours after exercise. In certain aspects the condition comprises taking nitrite and/or nitric oxide supplements. In some embodiments, supplements are nitrated fatty acids.

In certain embodiments, the steps are performed on multiple days and the intake of nitrate, nitrite, and/or related compounds is adjusted based on the measured test levels. In some cases, exposing the test substrate to saliva comprises indirectly exposing the saliva to the test substrate, such as providing the saliva to the test substrate by a finger or apparatus, or by spitting. In some cases, exposing the test substrate to an oral sample such as breath comprises breathing on the test substrate when the test substrate is outside of the oral cavity, inside the oral cavity but not touching the cavity, or both.

In certain aspects, the test substrate comprises a strip, disk, band, stick, swab, cup, vial, or string.

In particular aspects, the method is performed under conditions that prevent inaccurate measured test levels, such as conditions that comprise food or beverage intake without fasting, antibiotic intake or exposure, antiseptic oral care, diluted saliva, or a combination thereof. In specific cases, the individual has cardiovascular disease, obesity, diabetes, hypertension, atherosclerosis, hyperlipidemia, or is a smoker.

In some embodiments, there is a method of achieving a desired level of NO or nitrite in an individual, comprising the step of administering to the individual an effective amount of nitrite or nitrate when the individual has deficient levels as determined by providing an oral nitrite test substrate to the individual; and exposing said test substrate to an oral sample from the individual to produce a measured test level of NO or nitrite.

In some embodiments, there is a method for producing NO or nitrite in an individual according to the following reaction scheme:

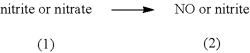

comprising the step of producing (2) in the individual when the level of (1) is deficient based on determination of (1) from the saliva or breath of the individual. In some cases, the determination of (1) from the saliva or breath of the individual comprises exposing the respective saliva or breath or other oral sample of the individual to a test substrate.

In certain embodiments, there is a method for detecting nitrite formation from nitrate-reducing bacteria in the oral cavity of an individual, comprising the step of providing a nitrate source to the individual; and measuring salivary levels of nitrite 1-3 hours later.

In some embodiments, there is a method of determining the level of nitric oxide (NO) or nitrite comprising the steps of providing a test substrate adapted to measure nitric oxide in an oral sample; receiving an oral sample on said test substrate; and indicating on the test substrate a measured level of NO or nitrite. The oral sample may be saliva, breath, or both.

In some embodiments of the invention, there is a composition comprising: a test substrate adapted to measure the nitric oxide activity of an oral sample, wherein said test substrate comprises: one or more indicia for indicating the level nitric oxide activity of an oral sample. In specific embodiments, the nitric oxide activity is correlated with one or more levels of nitrite, nitric oxide, nitrogen dioxide, dinitrogen trioxide (N₂O₃) dinitrogen tetroxide (N₂O₄) or nitrosating agents in the oral sample. The indicia may comprise colorimetric material. The test substrate may be formed of a strip, disk, band, stick, swab, cup, vial, powder, string or a combination. In some cases, the test substrate is confined within a package for commercial sale of nitric oxide supplements. In particular aspects, the test substrate is integrated with packaging for one or more nitric oxide supplements. In some cases, the methods and compositions are not for use with a urine sample and are not used with a urine sample.

In particular aspects of the invention, the substrate used to test an oral saliva sample can be wetted with an aqueous solution to test free nitric oxide gas.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 demonstrates factors that may obstruct salivary nitrite concentrations and subsequent NO availability

FIG. 2 provides studies testing NOx levels in patients undergoing hemodialysis.

FIG. 3 demonstrates salivary NOx levels in an individual following a single 4-5 hour dialysis session whereby 60-80% of the blood and plasma nitrite and nitrate is removed.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more elements or steps of the invention, for example. Some embodiments of the invention may consist of or consist essentially of one or more elements, method steps, and/or methods of the invention. It is contemplated that any method or composition described herein can be implemented with respect to any other method or composition described herein.

### I. General Embodiments of the Invention

The recognition of an enterosalivary circulation of nitrate and subsequent production of nitrite and nitric oxide provides a system for assessing nitric oxide activity and also a means to therapeutically intervene in conditions associated with nitric oxide insufficiency. In particular embodiments the methods and compositions of the invention ascertain bioavailability of endothelial NO. The present invention provides an individual with their current nitric oxide status, and use over time provides an individual with levels of NO over a period of time of use of the invention. The invention provides the advantage of encompassing a non-invasive method for assessing NO level without the need for seeing a medical provider to perform the method and analysis, given that the method is easy to use. The readout of the method for testing NO and nitrite levels is one that can be followed by action to address any insufficient levels of NO and nitrite in such an event. The individual can modify their activity or activities based on the method of the invention to increase NO level by ingesting foods higher in NO, exercising, and/or consuming active nitric oxide supplements, for example.

Certain embodiments of the present invention encompass methods and compositions related to a novel salivary nitrite test substrate that will allow rapid, non-invasive and semi-quantitative assessment of total body nitric oxide production/availability. As used herein, the term “semi-quantitative” refers to detection that can distinguish and differentiate the difference of a hundred or more micromolar concentrations in saliva or oral breath or other oral sample. The detected nitrite in the saliva or oral breath is a reflection of endogenous endothelial NO production as well as reduction of nitrate in the diet by oral commensal bacteria.

The methods of the invention may be performed on any individual of any age, gender, and sex.

In specific cases, the individual that uses methods and compositions of the invention is an individual that is known to have or suspected of having a dysfunctional eNOS/NO pathway or having endothelial dysfunction. The individual may have or be at risk of having cardiovascular disease. The individual may have or be at risk of having hyperlipidemia, diabetes, hypertension, atherosclerosis, inflammation, obesity, or be a smoker. The individual may have one or more risk factors for developing dysfunctional eNOS/NO pathway, endothelial dysfunction, or cardiovascular disease. Risk factors for such medical conditions are known in the art, including family history, smoking, high LDL, low HDL, hypertension, obesity, physical inactivity, High C-reactive protein, a combination thereof, and so forth.

### II. Measurement Methods of the Invention

The present invention provides useful methods and compositions for measuring and monitoring in vivo nitric oxide and nitrite levels in mammals, including in humans, dogs, cats, etc. The inventive methods measures nitric oxide activity including levels of nitrite, nitric oxide, nitrogen dioxide, dinitrogen trioxide (N₂O₃) dinitrogen tetroxide (N₂O₄) or any nitrosating agent in vivo in an individual. The measurement occurs in or from the mouth, such as from saliva of the mouth and/or from breath from the mouth. Any suitable means may be employed to measure the nitric oxide or nitrite orally. However, in specific embodiments the measurement occurs through a test substrate that houses or has applied thereto a certain chemical that allows detection of the nitric oxide or nitrite. Upon exposure to an oral sample, such as the saliva or breath, and its inherent nitric oxide or nitrite level therein, the chemical directly or indirectly generates a readout upon the test substrate that may be quantified to provide the level.

It is known that levels of salivary nitrite can vary between 50 μM up to over 2 mM (for example, the high range after nitrate rich meal). Exhaled NO levels also increase after a nitrate-rich meal. This broad range of nitrite concentrations can be detected on a test substrate using an assay of salivary or oral breath nitric oxide or nitrite. Because the enterosalivary circulation of nitrate is derived from both endogenous nitric oxide production as well as what is consumed through the diet, this pool of nitrite in the saliva or oral breath is a reflection of total body reservoir of nitric oxide and thus can be utilized as a novel NO diagnostic in humans.

In particular cases the methods of the invention are employed under conditions that do not result in false readings. In specific aspects, the methods are performed following a sufficient amount of time following exposure to conditions that would result in false readings. In particular cases, the methods are performed following a sufficient amount of time has occurred after consumption of food or beverage and prior to oral care, such as toothpaste or breath enhancers. In embodiments of the invention, the test substrate is employed after at least about a 3-5 hour fast (or longer, including overnight) to determine salivary or oral breath nitrite levels as a measure of total body NO availability. In specific embodiments, the methods are employed at least 3, 4, 5, 6, 7, 8, 9, 10, 11, or 12 hours or more following consumption of food or beverage.

Testing could be performed at any frequency, including at least once or twice daily, for example. In some embodiments, the method is performed once in the morning before eating and again at night 3-5 hours after consumption of the last food of the day. There are known circadian rhythms related to NO production, so testing throughout the day can provide more accurate assessment of NO production/availability, in at least certain cases.

The testing methods provide quantification of NO availability that may be interpreted based on a standard table of reference quantities. Ranges of NO availability can be assigned to the semi-quantitative table indicating, for example, “depleted”, “low” and/or “normal/optimal”. In specific embodiments, the correlation is as follows: depleted <20 μM; low 25-100 μM; normal 100-300 μM; and optimal >300 μM. In specific cases, an individual takes action to correct low nitric oxide or nitrite dependent on the outcome of the test. For example, an individual may take corrective measure(s) to increase nitrite or nitric oxide levels if the level is detected as too low, such as being low or depleted (or analogous labeling) following the test results.

Individuals could begin one or more regimens that can enhance NO production in the body and also monitor their NO status throughout the regimen(s). Moderate exercise and diets rich in foods with nitric oxide activity could acutely restore NO levels within a period of about 3-4 hours, or supplementing with nitric oxide drugs (e.g., nitroglycerin) or supplements (e.g., Neo40® of Neogenis of Austin Tex., U.S. patent application Ser. No. 12/484,364, filed Jun. 15, 2009 to Bryan, for example) would restore NO levels within 5-30 minutes, for example, and stabilize steady state salivary nitrite and breath NO in a period of weeks or months, for example.

In some embodiments of the invention, a supplementation composition is employed, comprising: a nitrite salt (for example, sodium or potassium) wherein said nitrite salt is provided in an amount ranging from about 10 mg to about 100 mg; 5 a nitrate salt (for example, sodium or potassium), wherein said nitrate salt is provided in an amount ranging from about 50 mg to about 500 mg; and ascorbic acid, wherein said ascorbic acid is provided in an amount ranging from about 100 mg to about 2000 mg; wherein said composition is provided in a single dose, including a single oral dose, in some cases. In some embodiments, the supplement composition comprises from about 1 weight part to about 8 weight parts nitrite, from about 5 weight parts to about 50 weight parts nitrate, and from about 20 weight parts to about 200 weight parts ascorbic acid. In certain embodiments, the supplement composition further comprises L-arginine, such as comprising from about 20 weight parts to about 200 weight parts L-arginine. In certain embodiments the supplement comprises water. In some embodiments of the supplement it is formulated to be exposed to commensal bacteria in the mouth. In specific embodiments, the supplement is solid. In particular embodiments a supplementation composition is provided in a kit with the test substrate.

In particular embodiments of the invention, an individual tests for NO levels before brushing their teeth or using any oral care product, including toothpaste, mouthwash, breath enhancers, breath mints, breath strips, and so forth (including of the antiseptic variety). In certain aspects of the invention, an individual tests for NO levels prior to consuming any food, beverage, taking any candy, or chewing any gum. In some aspects of the invention, an individual tests for NO levels prior to exercise. In specific cases wherein the individual has or needs to consume antibiotics, the test should be performed after sufficient time has passed from antibiotic intake, such as at least three days upon cessation of the antibiotic.

Any liquid taken prior to testing can dilute the saliva and cause “low” reading, so the individual should avoid beverages at least a half hour prior to testing, for example. Toothpastes can interfere with testing, and in particular sensitive toothpastes that contain potassium nitrate can interfere with the test and cause a false positive due to toothpaste. Certain infections by pathogenic bacteria in the mouth can also cause a false positive due to de-nitrification by these bacteria or because of the host immune response to the infection.

Although in some embodiments the saliva is placed directly on the test substrate, in other embodiments the saliva is exposed to the test substrate indirectly, such as by a finger (washed with soap, for example) or an apparatus, such as a stick or swab, for example.

In specific embodiments, the location of the assay region on the test substrate is exposed to water prior to use for the method. The water may be nitrate-free and nitrite-free in particular embodiments.

### III. Enhancement of Nitrite Levels Following Testing

In embodiments of the invention, an individual takes measures to increase in vivo nitrite levels following a result of insufficient nitrite with test methods of the invention. The individual may increase the levels in vivo by any suitable means, but in specific embodiments the individual can increase exercise, increase consumption of particular foods and/or beverages, and/or ingest nitrite drugs or supplements.

In particular cases one can increase their nitric oxide or nitrite levels by increasing intake of foods rich in nitrate or nitrite. Certain foods and beverages contain high levels of nitrate or nitrite compared to others. Green leafy vegetables (such as lettuce and/or spinach) and root vegetables (such as beetroot, radish, and/or carrots) contain high levels of nitrate that are converted to nitrites on ingestion. Pomegranates also are high in nitrates. Following ingestion of these exemplary foods or others that would raise nitrite or nitrate levels, one can measure the effectiveness of the individual diet at restoring NO homeostasis. Nitrites can also be found in cured meats, such as bacon and hot dogs; bologna, salami, corned beef, ham and sausages, and salmon and chicken that have been smoked. In some cases the individual can test for an increase in nitrite or nitrate levels following consumption of food or beverage without knowing whether or not the food or beverage had high nitrite or nitrate levels.

In particular embodiments, one tests for whether or not nitrite levels increased following consumption of certain foods either once or over time, but the testing is performed only after a period of fasting for a certain period of time, such as at least 3 or more hours including overnight, in specific embodiments. However, in alternative embodiments one can determine whether or not a food and/or beverage provided any NO activity to their body by utilizing the inventive test strips within about 90 minutes or more following the meal.

In certain embodiments one can increase nitric oxide or nitrite levels by increasing exercise. The exercise may be of any kind. In specific cases it comprises flexibility exercises, aerobic exercises, and/or anaerobic exercises. The exercise may encompass strength training; agility training; eccentric training; resistance training; interval training; and/or continuous training.

In some cases, one can increase nitric oxide or nitrite levels by supplementation. The supplementation may be of any kind, but in specific embodiments one can supplement orally or transdermally. The supplements may comprise nitrite, nitrate, nitric oxide, L-arginine, L-citrulline, or a combination thereof. The supplements may comprise hawthorne berry, bilberry, and/or beet root. The supplements may comprise one or more compositions that have nitrite reductase activity. The nitrite may comprise sodium nitrite and/or potassium nitrite, for example, or any cultured vegetable extract wherein nitrate has been reduced to nitrite. The nitrate may comprise sodium nitrate and/or potassium nitrate, for example or any naturally containing nitrate food such as green leafy vegetables or beet root. The supplements may comprise any of the inventions disclosed in U.S. patent application Ser. No. 12/484,364 by Bryan, filed Jun. 15, 2009, which inventions may be packaged or included with embodiments of the present invention disclosed herein.

In some cases, one can give a nitrate supplement or eat a nitrate-rich meal and then test approximately 90 minutes-3 hours later to determine the presence and activity of nitrate-reducing bacteria that will form nitrite in the saliva. The enterosalivary circulation of nitrate takes 1-3 hours and testing saliva within this time frame after a nitrate-rich meal provides a testing method for determining nitrate-reducing bacteria.

Some individuals may include two or more of the above-referenced means to increase nitrite levels, such as two or more of diet, exercise, and supplementation.

### IV. Test Substrates and Production Thereof

The present invention encompasses one or more test substrates for determining in vivo levels of nitric oxide and nitrite. The test substrate may be of any kind so long as it is capable of activity of identifying levels of nitric oxide or nitrite from an oral source, including saliva and/or breath. Substrates that can be detected include nitrite, nitrogen dioxide, dinitrogen trioxide (N₂O₃) dinitrogen tetroxide (N₂O₄) or any nitrosating agent, for example.

In specific embodiments, the test substrate is comprised of plastic, paper, a polymer, or any other material. The test substrate may be of any shape or configuration, but in at least specific cases the test substrate is a strip, disk, band, stick, swab, cup, vial, string, and so forth. In some cases the test substrate is confined within a package for commercial sale or on the outside of a package for commercial sale.

Any suitable chemical or chemicals may be present on the test substrate to detect NO or nitrite. In specific embodiments, the test substrate comprises 0.1-1.0% naphthylenediamine dihydrochloride and 0.5-5% sulphanilamide in 1-10% phosphoric acid. In specific embodiments, the test substrate comprises 1.0-10.0 mg p-Arsanilic Acid and 1.0-10.0 mg N-(1-naphthyl)ethylenediamine.

Production of the exemplary test substrates of the invention may occur by routine methods used in the art. For example, the blank test substrate may be exposed to the appropriate chemical at a specified location and allowed to dry or rest. In at least some cases the test substrates are allowed to dry in environments that are substantially free of NO, nitrate, nitrite, etc.

In some embodiments, the test substrate may comprise a means for testing other chemicals or conditions from the mouth. For example, one could also test for pH, ketone level, and/or protein level on the test substrate with the same or another oral sample from the individual.

In particular embodiments, the test substrate is placed in the mouth in direct contact with the tongue, such as in powder form. The color of the powder subsequent to placement in the mouth provides an interpretation of the in vivo level.

In some embodiments of the invention, one could actually discharge saliva, such as to spit, into a solution of the chemicals described herein and determine an output result, such as whether there is a change in color in a colorimetric assay, for example.

### V. Exemplary Kits of the Invention

Any of the compositions described herein may be comprised in a kit. In a non-limiting example, a kit in suitable container means is provided that allows testing of the level of measuring oral (including salivary or breath) nitrite or NO. In specific embodiments, the kit comprises reagents and/or substrates for testing nitrite, nitrogen dioxide, dinitrogen trioxide (N₂O₃) dinitrogen tetroxide (N₂O₄), nitric oxide, or any nitrosating agent.

The kits may comprise a suitably packaged reagent and/or substrate composition of the present invention. The components of the kits may be packaged suitably to avoid contamination or destruction of the reagents and/or substrates prior to use. The reagents and/or substrates may be individually packed, including individually wrapped, or they may be packaged having multiple quantities. Where there are more than one component in the kit, the kit also will generally contain a second, third or other additional container into which the additional components may be separately placed. The kits of the present invention also will typically include a means for containing the reagents and/or substrates in close confinement for commercial sale. Such containers may include injection or blow-molded plastic containers into which the desired reagents and/or substrates are retained. The test substrates may be packaged in the kit with multiple substrates in a vial.

In specific embodiments, the kit provides literature that gives information on how to interpret readouts for detected levels of nitrite or NO. The legend may be included a separate piece of paper or may be on the packaging, or both. The legend may be present on the outside of a vial, for example.

The kits of the present invention will also typically include a means for containing the vials in close confinement for commercial sale, such as, e.g., injection and/or blow-molded plastic containers into which the desired vials are retained.

In some embodiments of the present invention, the kit is provided with a test substrate and a composition for raising in vivo levels of NO or nitrite. For example, in addition to the test substrate(s) the kit may comprise one or more nitrite, nitrate, nitrated fatty acids, or NO supplements.

In some embodiments, the test substrates are packaged inside a vial or other package that lacks an outer container.

In some cases the test substrate is confined within a package for commercial sale or on the outside of a package for commercial sale

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Method of Diagnosing, Correcting and Measuring NO Levels in Humans

One can determine salivary nitrite levels using novel test strips. Individuals could apply saliva to the test strip and get a readout within at least about 3 seconds.

Individuals may begin a regimen of NO-boosting activities including exercise, taking active nitric oxide supplements, and/or including diets rich in NO activity (foods high on the NO index).

One can continue to monitor salivary nitrite as a measure of total body NO availability daily, weekly, or monthly, but in certain embodiments it is daily or weekly.

The present invention provides a means of self monitoring individual health as it relates to NO. With NO involved in virtually every biological system, this approach has a profound effect on public health and disease prevention by correcting NO insufficiency before the manifestation of disease and symptoms.

### Example 2

### Measuring In Vivo Nitrite Levels

An exemplary confirmation of the present invention involved using test strips from Cenogenics Inc, (Morganville, N.J.) a company that manufactures urinary test strips for diagnostic purposes. The test pad for nitrite detection contains p-Arsanilic Acid—5.0 mg and N-(1-naphthyl)ethylenediamine—6.0 mg. This detection principle is based on the Griess Reaction. The method of the present invention was validated for detecting nitrite in saliva.

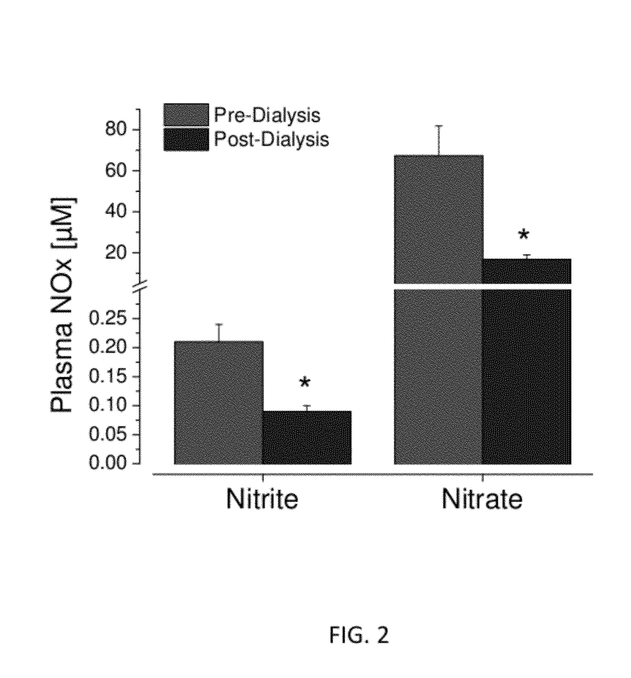

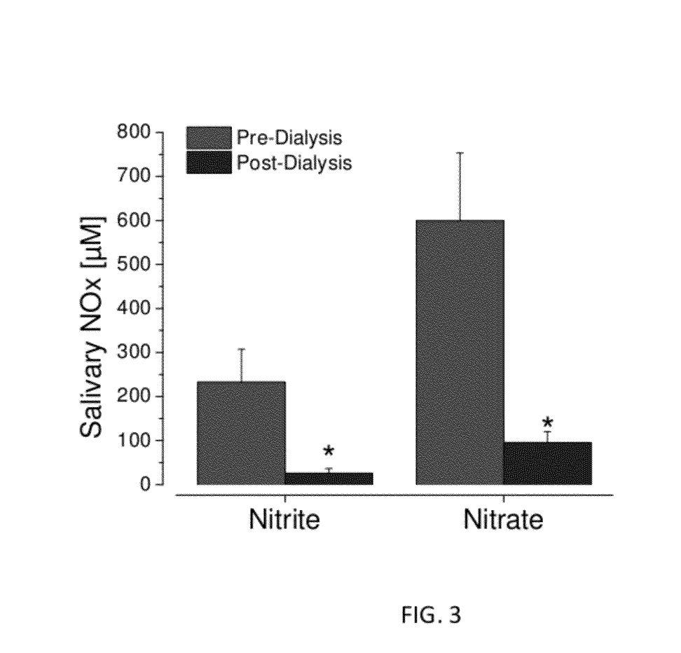

To demonstrate that changes in blood levels are also reflected in saliva, studies on patients undergoing hemodialysis were performed. The hemodialysis process removes all water soluble anions that are not in the dialysate solution and causes some degree of endothelial dysfunction. Data shown in FIG. 2 reveal that hemodialysis effectively removes blood and plasma nitrite and nitrate in patients with end stage renal disease. It is well-established that patients on hemodialysis have a higher mortality than age-matched patients not on dialysis primarily due to higher incidence of cardiovascular related deaths. Loss of bioactive NO through the hemodialysis process may be responsible for this increased incidence of cardiovascular accidents. The data in FIG. 3 reveal that a single 4-5 hour dialysis session whereby 60-80% of the blood and plasma nitrite and nitrate is removed, salivary levels of nitrite and nitrate are reduced to an even greater extent by 90%. These data demonstrate that salivary nitrite may be a more reliable and sensitive measure of total body NO availability than blood markers.

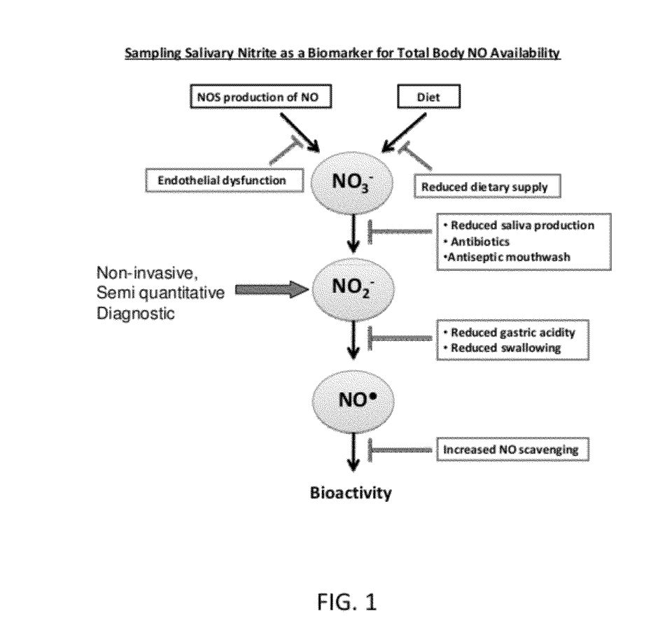

As a result of this human nitrogen cycle and enterosalivary circulation of nitrate (both from diet and from endogenous NO production) and subsequent reduction to nitrite in the mouth, sampling salivary nitrite can be used as an accurate representation of total body NO production/availability. This embodiment is illustrated in FIG. 1.

The skilled artisan recognizes that people may not have the same communities of oral bacteria that are responsible for reducing salivary nitrate to nitrite. For example, a population that uses antiseptic mouthwash would have a different degree of oral hygiene compared to a population that does not use antiseptic mouthwash. Also, steady state concentrations of nitrite in the saliva are dependent upon salivary flow rates and production. Patients with Sjogren's syndrome or parotidectomy due to cancer or other conditions may have a breakdown in this pathway and may not have an accurate assessment.

Testing may be done when fasted and no earlier than one hour after drinking any liquid beverage to prevent dilution of the saliva and a false negative reading. Understanding the basis and rationale for sampling salivary nitrite as well as recognizing the limitations, this non-invasive diagnostic can be an accurate assessment of total body NO availability and provide new patient information to cardiovascular risk and NO homeostasis. The ability to diagnose, correct and monitor NO status in patients imparts a profound effect on their health and well-being.

### Example 3

### Determination of NO Gas in Oral Breath

One can determine NO gas in oral breath using novel test strips. Individuals could wet test strip with nitrite-free water and then place the strip in the mouth (not touching any part of the mouth or tongue) and allow the gas in the mouth to equilibrate with the test strip. Free NO gas will react with oxygen in the mouth to form a nitrosating species that will activate the test strip (similar to nitrite). This method requires 1-2 minutes of equilibration for accurate assessment of free NO in the mouth, in specific embodiments.

### Example 4

### Determination of Nitrate Reducing Bacteria in the Oral Cavity

One can determine the presence and/or activity of nitrate-reducing bacteria in the oral cavity after a nitrate-rich meal or after giving a nitrate supplement. Certain commensal bacteria possess nitrate reductase enzymes that can reduce salivary and oral nitrate to nitrite. The presence and activity of these bacteria contribute nitrite and nitric oxide to the host. Determining the presence of nitrate-reducing bacteria is useful as a novel risk factor for at least NO insufficiency and cardiovascular disease, for example. One could test salivary nitrite 1-3 hours after a nitrate load to assess salivary nitrite formation from nitrate-reducing bacteria.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of determining the level of nitric oxide (NO) or nitrite comprising the steps of:
providing a test substrate adapted to measure nitric oxide in an oral sample;
receiving an oral sample on said test substrate; and
indicating on said test substrate a measured level of NO or nitrite.

2. The method of claim 1, wherein said oral sample is saliva.

3. The method of claim 1, wherein said oral sample is breath.

4. The method of claim 1, further comprising the step of correlating the measured test level with in vivo NO or nitrite level in the individual.

5. The method of claim 1 wherein the method is performed after fasting for at least three hours.

6. The method of claim 1 wherein the measured test level is generated with colorimetric indicia.

7. The method of claim 1, wherein the method is performed for the individual more than once.

8. The method of claim 1, wherein the method is performed for the individual at regular intervals.

9. The method of claim 8, wherein said regular intervals are once or twice daily measurements.

10. The method of claim 1, wherein the method is performed following subjecting the individual to conditions that increase the in vivo level of NO or nitrite.

11. The method of claim 10, wherein the condition comprises consumption of a nitrate-rich food and/or beverage.

12. The method of claim 11, wherein the method is performed at least 1.5 hours after consumption of the food and/or beverage.

13. The method of claim 10, wherein the condition comprises exercise.

14. The method of claim 13, wherein the method is performed at least three hours after exercise.

15. The method of claim 10, wherein the condition comprises taking one or more supplements comprising a nitrite, nitrate, and/or nitrate reductase.

16. The method of claim 15, wherein the supplements are nitrated fatty acids.

17. The method of claim 4, wherein the steps are performed on multiple days and the intake of nitrate, nitrite, and/or related compounds is adjusted based on the measured test levels.

18. The method of claim 1, wherein exposing the test substrate to saliva comprises indirectly exposing the saliva to the test substrate.

19. The method of claim 18, wherein the saliva is provided to the test substrate by a finger, by apparatus, or by spitting.

20. The method of claim 1, wherein exposing the test substrate to breath comprises breathing on the test substrate when the test substrate is outside of the oral cavity, inside the oral cavity but not touching the cavity, or both.

21. The method of claim 1, wherein the test substrate is a strip, disk, band, stick, swab, cup, vial, or string.

22. The method of claim 1, wherein the method is performed under conditions that prevent inaccurate measured test levels.

23. The method of claim 22, wherein the conditions comprise food or beverage intake without fasting, antibiotic intake or exposure, antiseptic oral care, diluted saliva, or a combination thereof.

24. The method of claim 1, wherein the individual has cardiovascular disease, obesity, diabetes, hypertension, atherosclerosis, hyperlipidemia, or is a smoker.

25. A method of achieving a desired level of nitric oxide activity in an individual, comprising the steps of:
administering nitrites or nitrates to an individual;
receiving on a test substrate saliva or breath from the individual; and
indicating on said test substrate a measured test level nitric oxide activity of the saliva or breath from the individual.

26. A method for producing NO or nitrite in an individual according to the following reaction scheme: 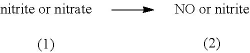
comprising the step of producing (2) in the individual when the level of (1) is deficient based on determination of (1) from the saliva or breath of the individual.

27. The method of claim 26, wherein the determination of (1) from the saliva or breath of the individual comprises exposing the respective saliva or breath of the individual to a test substrate.

28. A method for detecting nitrite formation from nitrate-reducing bacteria in the oral cavity of an individual, comprising the step of:
providing a nitrate source to the individual; and
measuring salivary levels of nitrite 1-3 hours later.

29. A composition comprising:
a test substrate adapted to measure the nitric oxide activity of an oral sample, wherein said test substrate comprises:
one or more indicia for indicating the level nitric oxide activity of an oral sample;

30. The composition of claim 29, wherein the nitric oxide activity is correlated with one or more levels of nitrite, nitric oxide, nitrogen dioxide, dinitrogen trioxide (N₂O₃) dinitrogen tetroxide (N₂O₄) or nitrosating agents in the oral sample.

31. The composition of claim 29, wherein the indicia comprises colorimetric material.

32. The composition of claim 29, wherein the test substrate is formed of a strip, disk, band, stick, swab, cup, vial, powder, string or a combination.

33. The composition of claim 29 wherein the test substrate is confined within a package for commercial sale of nitric oxide supplements.

34. The composition of claim 29 wherein the test substrate is integrated with the packaging for one or more nitric oxide supplements.
